(19) United States
(12) Patent Application Publication
(10) Pub. No.: US 2025/0049868 A1
(43) Pub. Date: Feb. 13, 2025

(54) DRUG AND HEALTH FOOD FOR SUPPRESSING GENE MUTATION

(71) Applicant: FORDAYS CO., LTD., Tokyo (JP)

(72) Inventors: Keisuke KIRIYAMA, Tokyo (JP); Kunio MATSUMOTO, Ishikawa (JP); Keita SUTOH, Tokyo (JP); Katsuya SAKAI, Ishikawa (JP); Ryu IMAMURA, Ishikawa (JP); Hiroki SATO, Ishikawa (JP)

(21) Appl. No.: 18/262,759

(22) PCT Filed: Jan. 25, 2022
(86) PCT No.: PCT/JP2022/002729

§ 371 Date: Feb. 12, 2024

(30) Foreign Application Priority Data
Jan. 26, 2021 (JP) 2021-010644

(51) Int. Cl. A61K 36/064; A61K 9/00; A61K 31/7105; A61K 47/46; A61P 43/00

(52) U.S. Cl. CPC A61K 36/064; A61K 9/0056; A61K 31/7105; A61K 47/46; A61P 43/00

(57) ABSTRACT

A drug and a health food for suppressing gene mutations using nucleic acids and RNA. The drug or the health food contains a yeast extract and one or more components derived from a plant, fish, and a dairy product. The yeast extract is present as an active ingredient and particularly contains the RNA as an active ingredient. It does not inhibit DNA damage, but promotes an action of repairing damaged DNA or promotes an action of killing cells containing damaged DNA to suppress gene mutations.

### TECHNICAL FIELD

The present invention relates to a drug and a health food for suppressing gene mutations. In detail, the present invention relates to a drug and a health food that contain a component derived from a plant, fish, and a dairy product as an active ingredient, particularly RNA as an active ingredient, in addition to a yeast extract to suppress gene mutations. Note that the health food includes a beverage.

### BACKGROUND ART

Gene mutations contribute to a variety of diseases including a cancer. Therefore, suppressing gene mutations probably leads to prophylaxis of many diseases.

Each one of cells that makes up a body of an organism internally includes nuclei, and a nucleus internally contains DNA. DNA is easily damaged due to environmental factors, and the environmental factors include ultraviolet ray, active oxygen, chemical substances, and the like. For example, exposure of DNA to ultraviolet ray dimerizes two adjacent bases (for example, thymine-thymine), resulting in DNA damage. DNA damage predisposes to a replication error in the portion during DNA replication, and gene mutations occur from the portion. Another example is that when a chemical substance, ethylnitrosourea (N-Nitroso-N-ethylurea: ENU), enters an inside of a body, the ENU directly acts on DNA to cause alkylation, and this induces DNA damage to cause gene mutations.

However, DNA damage does not always result in gene mutations. A body of an organism has a DNA damage repair mechanism, and an action of a DNA repair enzyme or the like repairs a DNA damage site before DNA replication. It is said that several tens of thousands of DNAs are damaged per cell in a day in case of a human, but most of them are repaired and do not lead to gene mutations. It is considered that, in a case where DNA damage is beyond the repair ability, the repair cannot catch up, and DNA replication occurs with the DNA damage left, thus casing gene mutations.

Up to the present, there have been several reports on suppression of DNA damage caused by ingestion of food or the like. However, these studies targeted DNA damage, and it is unknown whether suppression of the DNA damage leads to suppression of gene mutations. Regarding studies involving nucleic acids, an in-vitro test in which addition of polymer DNA promotes the repair of DNA damage after ultraviolet irradiation (Non-Patent Document 1) is reported, and an in-vivo test in which DNA damage of a mouse caused by administration of cyclophosphamide is suppressed by ingestion of ribonucleotide (Non-Patent Document 2) is reported. Additionally, an in-vitro test in which addition of small nuclear protein (DNA and protein) suppresses oxidative damage of DNA (Patent Document 1) is reported. However, as described above, these studies also target DNA damage, and whether the actions lead to suppression of gene mutations is unknown.
Patent Document 1: JP-A-2003-313130
Non-Patent Document 1: Photodermatol Photoimmunol Photomed 2007; 23:242-249
Non-Patent Document 2: Journal of Animal Physiology and Animal Nutrition 92 (2008) 211-218

### DISCLOSURE OF THE INVENTION

### Problems to be Solved by the Invention

The present inventors determined to address elucidation of the mechanism of suppression of gene mutation. An object of the present invention is to provide a drug and a health food that suppress gene mutations.

The present inventors have perceived that a yeast extract and components derived from a plant, fish, and a dairy product (hereafter also referred to as a yeast extract or the like), exert a suppressive effect on gene mutations, particularly RNA contained in a yeast extract or the like exerts a suppressive effect on gene mutations. Based on this, the drug and the health food that suppress gene mutations have been completed as the present invention.

### Solutions to the Problems

The ENU is known to have more potent mutagenicity than other mutagenic compounds and to induce gene mutations in various organs throughout a body. Therefore, the present inventors selected intraperitoneal administration of ENU as a method for inducing gene mutations, and verified a suppressive effect on gene mutations by ingestion of a yeast extract or the like (RNA contained in the yeast extract) using a test method referred to as Pig-a assay that can examine a frequency of gene mutations from a trace amount of peripheral blood collected from mice and the like. The present inventors have found the suppressive effect on gene mutations by the ingestion of RNA from results of the test method referred to as Pig-a assay.

In addition, the point of this test is that the RNA contained in the yeast extract or the like was ingested in the original form of RNA (the form originally present in food). While the report of Non-Patent Document2 made it clear that the DNA damage of the mouse was suppressed (defense against the damage) by ingesting monoribonucleotide. However, normally, ribonucleotide exists in food in the form of RNA (polymer, the form of the polymer), not a monomer. The present inventors have seen that the effects of the mutagenic compounds change depending on the amount of RNA in the diet of the mouse through ingestion in the form of RNA at this time and have found the suppressive effect on gene mutations in a manner similar to what is called RNA ingested in our lives.

The present invention has been completed based on the above-described perception.

That is, one aspect of the present invention relates to the following.
1. A drug for suppressing a gene mutation contains a yeast extract and RNA. The RNA included in the yeast extract is contained as an active ingredient.
2. The drug for suppressing the gene mutation according to 1 further contains one or more of components and RNA. The one or more of components are derived from a plant, fish, and a dairy product in addition to the yeast extract. The RNA is contained as an active ingredient.
3. In the drug for suppressing the gene mutation in accordance with 1 or 2, the RNA promotes an action of repairing damaged DNA or promotes an action of killing cells containing the damaged DNA.
4. In the drug for suppressing the gene mutation in accordance with any one of 1 to 3, the RNA is contained by 0.08 to 20 mass %.
5. In the drug for suppressing the gene mutation in accordance with any one of 1 to 4, the RNA contained in the yeast extract is RNA extracted from a torula yeast.
6. A health food for suppressing a gene mutation contains a yeast extract and RNA. The RNA included in the yeast extract is contained as an active ingredient.
7. The health food for suppressing the gene mutation in accordance with 6, further contains one or more of components and RNA. The one or more of components are derived from a plant, fish, and a dairy product in addition to the yeast extract. The RNA is contained as an active ingredient.
8. In the health food for suppressing the gene mutation in accordance with 6 or 7, the RNA promotes an action of repairing damaged DNA or promotes an action of killing cells containing the damaged DNA.
9. In the health food for suppressing the gene mutation in accordance with any one of 6 to 8, the RNA is contained by 0.08 to 20 mass %.
10. In the health food for suppressing the gene mutation in accordance with any one of claim 6 to claim 9, the RNA contained in the yeast extract is RNA extracted from a torula yeast.

Additionally, another aspect of the present invention relates to a method for suppressing a gene mutation by oral administration of the drug and the health food that suppress the gene mutation to a target and further relates to use of the drug and the health food that suppress the gene mutation for suppressing the gene mutation.

### Effects of the Invention

According to the present invention, the drug and the health food that suppress the gene mutations, are considerably safe, and have few side effects are provided.

The drug and the health food that suppress the gene mutations of the present invention can suppress the gene mutations by promoting an action of repair when DNA is damaged or promoting an action of killing cells containing damaged DNA.

### BRIEF DESCRIPTION OF THE DRAWINGS

FIG. 1 is a graph in which body weights of mice during an experimental period were measured.

FIG. 2 is a diagram illustrating examples of measurement results of Pig-a assay using a flow cytometer.

FIG. 3 is a graphical showing Pig-a gene mutation frequency after administration of ENU to mise during the experimental period.

### DESCRIPTION OF PREFERRED EMBODIMENTS

The following will describe the present invention in further detail.

Nucleic acid is a generic term for deoxyribonucleic acid (DNA) that holds genetic information and ribonucleic acid (RNA) that transmits the genetic information and synthesizes proteins following the information that the DNA has and is a considerably important substance to maintain vital activities, in addition to have an important role for proliferation and growth of cells. One of food components described in the present invention contains a high amount of nucleic acids of torula yeast. Among food products, torula yeast contains a particularly high amount of nucleic acids. Torula yeast is a yeast approved by the U.S. Food and Drug Administration (FDA) for safety as food. Fungus bodies are produced using sugar, such as a pulp effluent and blackstrap molasses. Nucleic acids (RNA) extracted from the fungus body have been utilized as a health food. In the present invention, as well as examining effects of a torula yeast extract on gene mutations, in addition to a yeast extract other than torula yeast, an active ingredient contained in components derived from a plant, fish, and a dairy product, particularly RNA was examined for effects given to gene mutations.

Gene mutations are considered to be caused by damage to genes due to ultraviolet ray, active oxygen, chemical substances in an environment, or the like, resulting in expression of abnormal proteins and causing a variety of diseases. Regarding a cancer, accumulation of gene mutations is believed to induce cancerization. In particular, mutations in genes that regulate cell division are considered to result in a cancer, and thus suppression of DNA damage and gene mutations is considered to be crucial to maintain health.

To suppress the gene mutations, organisms have several defenses. One is a repair reaction of DNA damage, and the second is an apoptotic reaction dependent on mismatch repair proteins. A possibility of involvement of autophagy in repairing DNA damage is also conceivable.

The drug and the health food that suppress gene mutations in one aspect of the present invention use the yeast extract, a component derived from a plant, such as a corn, wheat, soybeans, and rice, and a component derived from fish and a dairy product, but the components are not limited thereto. Examples of the yeast include brewer's yeast, torula yeast, milk yeast, and baker's yeast, and extracts from these yeasts (particularly RNA) can be used.

The active ingredient in the drug and health goods that suppress gene mutations of the present invention is the yeast extract or the like, and particularly RNA is conceivable. Hereinafter, the description of a “gene mutation suppressive substance” represents the yeast extract or the like.

The administration forms of the drug that suppresses the gene mutations of the present invention include injections (subcutaneous, intravenous, intramuscular, and intraperitoneal injection), parenteral administration with ointments, suppositories, aerosols, and the like, and oral administration with tablets, capsules, granules, pills, syrups, solutions, emulsions, suspension agents, and the like.

The drug that suppresses the gene mutations of the present invention contains RNA as the gene mutation suppressive substance about 0.01 to 99.5 mass %, preferably about 0.05 to 50 mass %, and more preferably about 0.08 to 20 mass % per the mass of all compositions.

In addition to the gene mutation suppressive substance as the active ingredient, the drug that suppresses the gene mutations of the present invention can also contain other pharmaceutically or veterinarily active compounds.

The clinical dosage of the gene mutation suppressive substance contained in the drug that suppresses the gene mutations of the present invention differs depending on an age, a body weight, sensitivity of a patient, a degree of symptoms, and the like. The dosage of RNA of the gene mutation suppressive substance contained in the drug or the health food that suppresses the gene mutations is 0.01 mass % or more, preferably 0.05 mass % or more, and more preferably 0.08 mass % or more per a mass of a meal normally ingested at a time. However, when necessary, an amount outside the above-described ranges can be used.

A gene mutation retarder of the present invention is formulated for administration by conventional pharmaceutical means.

Thus, tablets, capsules, granules, and pills for oral administration are prepared using: excipients, such as sucrose, lactose, dextrose, starch, and mannitol; binders, such as hydroxypropylcellulose, syrup, gum arabic, gelatin, sorbitol, tragacanth, methylcellulose, and polyvinylpyrrolidone; disintegrators, such as starch, carboxymethylcellulose or its calcium salt, microcrystalline cellulose, and polyethylene glycol; glidants, such as talc, magnesium stearate or calcium, and silica; lubricants, such as sodium laurate and glycerol, and the like.

Injections, solutions, emulsions, suspensions, syrups, and aerosols are prepared using: solvents of active components, such as water, ethyl alcohol, isopropyl alcohol, propylene glycol, 1,3-butylene glycol, and polyethylene glycol; surfactants, such as sorbitan fatty acid ester, polyoxyethylene sorbitan fatty acid ester, polyoxyethylene fatty acid ester, polyoxyethylene ether of hydrogenated castor oil, and lecithin; suspensions, such as carboxymethyl sodium salt, cellulose derivative, such as methyl cellulose, tragacanth, and natural gums, such as gum arabic; and preservatives, such as esters of parahydroxybenzoic acid, benzalkonium chloride, and sorbate, and the like.

As the ointment, which is a transdermal absorption preparation, for example, white petrolatum, liquid paraffin, higher alcohol, macrogol ointment, hydrophilic ointment, aqueous gel base, and the like are used.

The suppository is prepared using, for example, cacao butter, polyethylene glycol, lanolin, fatty acid triglyceride, coconut oil, polysorbate, and the like.

The following shows examples of formulations of the drugs that suppress gene mutations of the present invention.

### Example of Formulation 1

Tablets

| | | |
|---|---|---|
| RNA in the gene mutation suppressive substance | 1 to | 30 g |
| Lactose | 200 to | 300 g |
| Microcrystalline cellulose | 500 to | 700 g |
| Cornstarch | 300 to | 400 g |
| Hydroxypropyl cellulose | 50 to | 200 g |
| CMC-Ca | 100 to | 200 g |
| Magnesium stearate | 10 to | 60 g |

| | |
|---|---|
| Total amount | 1,500 g or more(*) |

After the components are mixed by the conventional method, 10,000 sugar-coated tablets are produced.
(*) The total amount is increased by the percentage of the RNA in the gene mutation suppressive substance (yeast extract). The same applies to the “total amount” below.

### Example of Formulation 2

Capsules

| | | |
|---|---|---|
| RNA in the gene mutation suppressive substance | 1 to | 30 g |
| Lactose | 300 to | 500 g |
| Microcrystalline cellulose | 500 to | 1200 g |
| Magnesium stearate | 10 to | 100 g |

| | |
|---|---|
| Total amount | 1,500 g or more |

After the components are mixed by the conventional method, the product is filled in gelatin capsules to produce 10,000 capsules.

### Example of Formulation 3

Soft capsules

| | | |
|---|---|---|
| RNA in the gene mutation suppressive substance | 2 to | 40 g |
| PEG400 | 400 to | 500 g |
| Saturated fatty acid triglyceride | 1000 to | 2000 g |
| Mentha oil | 0.2 to | 2 g |
| Polysorbate 80 | 5 to | 20 g |

| | |
|---|---|
| Total amount | 2,000 g or more |

After the components are mixed by the conventional method, the product is filled in No. 3 soft gelatin capsules to produce 10,000 soft capsules.

### Example of Formulation 4

Ointments

| | | |
|---|---|---|
| RNA in the gene mutation suppressive substance | 0.1 to | 3 g |
| Liquid paraffin | 5 to | 20 g |
| Cetanol | 10 to | 30 g |
| White petrolatum | 60 to | 80 g |
| Ethylparaben | 0.05 to | 3 g |
| 1-menthol | 0.2 to | 2 g |

| | |
|---|---|
| Total amount | 100.0 g or more |

The components are mixed by the conventional method to produce ointments.

### Example of Formulation 5

Suppositories

| | | |
|---|---|---|
| RNA in the gene mutation suppressive substance | 1 to | 30 g |
| Witepsol H15* | 400 to | 600 g |
| Witepsol W35* | 500 to | 600 g |
| Polysorbate 80 | 0.5 to | 21 g |

| | |
|---|---|
| Total amount | 1,000 g or more |

″*Witepsol as a trade name of a triglyceride compound.″

The components are melted and mixed by the conventional method, poured into a suppository container, and cooled and solidified to produce 1,000 suppositories.

### Example of Formulation 6

Injections

| | |
|---|---|
| RNA in the gene mutation suppressive substance | 1 to 15 mg |
| Distilled water for injection | 5 mL |

In use, the injection is dissolved for use.

The present invention also relates to a health food containing the gene mutation suppressive substance. The active ingredient in the health food of the present invention is the gene mutation suppressive substance, that is, the yeast extract or the like and particularly RNA.

The health food of the present invention, for example, is preferably embodied as a health food having a gene mutation suppressing action. In addition, a product may fit user's preference by mixture with various components, such as the known sweetener, acidulant, and vitamin. For example, it is possible to provide it in the form of a dairy product, such as a tablet, a capsule, a drinkable preparation, a jelly, and a yogurt, a seasoning, a processed food, a supplement, desserts, a confectionery, and the like.

Although there is no particular limitation on the producing process of these health foods, for example, an objective health food can be produced by adding the above-described sweetener or the like by appropriate means during processing of the health food. The gene mutation suppressive substance can be combined in a range around 1 mg to 20 g or 0.08 g to 20 g per 100 g of the food.

Specific substances that can be added to the health food of the present invention include the following, but are not limited to the following.

As a milt extract, after skin, muscle, blood vessel, and the like are removed from a milt, purification is performed to remove oil content, and an enzymatic decomposition process is performed with nuclease and protease to allow producing water-soluble nucleoprotein. As the milt, for example, milts of salmon, trout, and herring can be used.

Examples of collagen include porcine collagen peptide, fish collagen peptide (including gelatin), and a collagen-containing mineral complex. The collagens described above can be used alone or can also be used as a mixture of two or more kinds of them.

The chondroitin includes a chondroitin, which is a kind of glycosaminoglycan (mucopolysaccharide) and has a basic structure in which sulfuric acid is linked to a sugar chain containing two alternating monosaccharides: D-glucuronic acid (GlcA) and N-acetyl-D-galactosamine (GalNAc), derivatives thereof and salts thereof, which are referred to as chondroitins.

The hyaluronic acid includes a hyaluronic acid, which is a kind of proteoglycan and has a basic structure of linked units of disaccharide in which the 1st position of β-D-glucuronic acid and the 3rd position of β-D-N-acetyl-glucosamine are bonded, derivatives thereof and salts thereof, which are referred to as hyaluronic acids, and low molecular hyaluronic acid or hyaluronic acid decomposition products obtained by treating such hyaluronic acids with an enzyme, such as hyaluronidase, or by subjecting such hyaluronic acids to heating and pressurizing treatment. The specific hyaluronic acid that can be added to health foods includes a rooster comb extract and the like.

As long as the arginine can be added in an aspect that can be used for a food, the arginine is not particularly limited, and an aspect of arginine alone and an aspect in which arginine molecules and acid molecules are linked are included.

As long as the magnesium carbonate is also an ethical drug and can be added in an aspect usable for foods, the magnesium carbonate is not particularly limited. As the magnesium salt, instead of magnesium carbonate or a part thereof, magnesium oxide, magnesium chloride, or the like may be added.

As long as the zinc can be added in an aspect usable for foods, the zinc is not particularly limited, and can be administered in an aspect, such as zinc gluconate, zinc sulfate, or edible zinc yeast.

The vitamins are not particularly limited as long as they are vitamins, derivatives thereof, or salts thereof, which can render the effects of the present invention. Examples include vitamin C (ascorbic acid), vitamin B1 (thiamine), vitamin B2 (riboflavin), vitamin B6 (pyridoxine), vitamin B12 (cobalamin), folic acid (vitamin B9), niacin (vitamin B3), and calcium pantothenate.

Examples of the other components include a sweetener, such as fructose glucose liquid sugar, superfine sugar, rare sugar-containing syrup, erythritol, and sucralose, a fruit juice, such as pineapple fruit juice, a preservative, such as sodium benzoate, a coloring agent, such as a caramel pigment, an emulsifier (derived from soybeans, for example), a flavor, and an acidulant, and when the health food of the present invention contains these other components, the appropriate amount of each component can be added.

The following shows examples of the components of the health food of the present invention.

### Example of Health Food 1

Beverage (amount per 720 mL)

| | | |
|---|---|---|
| RNA in the gene mutation suppressive substance | 0.6 g to | 18 g |
| Milt extract | 4000 to | 4500 mg |
| Collagen | 60 to | 90 g |
| Chondroitin | 100 to | 200 mg |
| Hyaluronic acid | 50 to | 80 mg |
| Arginine | 1500 to | 2000 mg |
| Magnesium carbonate | 1000 to | 2000 mg |
| Zinc | 30 to | 40 mg |
| Folic acid | 2 to | 4 mg |
| Niacin | 100 to | 200 mg |
| Vitamin C | 3000 to | 4000 mg |
| Vitamin B1 | 13 to | 14 mg |
| Vitamin B2 | 14 to | 15 mg |
| Vitamin B6 | 15 to | 16 mg |
| Vitamin B12 | 25 to | 27 mg |
| Calcium pantothenate | 70 to | 90 mg |

### Other Additional Ingredients Appropriate Amount

(Other additional ingredients: fructose glucose liquid sugar/superfine sugar/rare sugar-containing syrup/erythritol/pineapple juice, and the like)

### Example of Health Food 2

Jelly (amount per 15 g)

| | | |
|---|---|---|
| RNA in the gene mutation suppressive substance | 10 to | 300 mg |
| Milt extract | 100 to | 200 mg |
| Zinc | 1 to | 2 mg |
| Vitamin C | 60 to | 90 mg |
| Vitamin B1 | 0.7 to | 0.9 mg |
| Vitamin B2 | 1.0 to | 1.2 mg |
| Vitamin B6 | 0.8 to | 1.0 mg |

Other additional ingredients Appropriate amount (Other additional ingredients: collagen/chondroitin/hyaluronic acid/sweetener/vitamin B12/fruits puree, and the like)

### Example of Health Food 3

Capsule supplement (amount per 12 capsules)

| | | |
|---|---|---|
| RNA in the gene mutation suppressive substance | 80 to | 2400 mg |
| Milt extract | 500 to | 700 mg |
| Arginine | 400 to | 600 mg |
| Zinc | 6 to | 9 mg |
| Vitamin C | 400 to | 600 mg |
| Copper | 0.4 to | 0.6 mg |

Other additional ingredients Appropriate amount

(Other additional ingredients: brewer's yeast/Ginkgo biloba extract/odorless garlic extract/porcine placenta extract/dextrin/selenium/gelatin/sucrose fatty acid ester/sodium ferrous citrate/calcium pantothenate/vitamin B1/vitamin B2/vitamin B12/vitamin A/vitamin D/folic acid, and the like)

### EXAMPLES

Hereinafter, one aspect of the present invention will be specifically described with reference to Examples, but the present invention is not limited to the following examples. Mice, samples, and the like used in the following examples are as follows.

### 1. Mouse Breeding

Five-week old male C57BL/6J mice were introduced, classified into three groups considering that their body weights were averaged, and fed experimental feeds after habituation. ENU was administered intraperitoneally on day 15 of feeding experimental feed. The experimental feeds were continuously fed as well after the administration of the ENU.

The food intakes from day 8 to day 10 of feeding the experimental feed and the food intakes from day 20 to day 22 after the ENU administration were measured.

The body weights were measured weekly from the start of feeding the experimental feed.

The bloods were collected from the tail veins on day 8, day 15, day 22, day 29, and day 43 after the ENU administration and used for Pig-a assay.

### 2. Materials and Methods

### 2-1. Experimental Animals and Breeding Environments

The five-week old male C57BL/6J mice were purchased from CLEA Japan, Inc. and maintained in a barrier breeding room in the Utsunomiya office of Institute of Immunology Co., Ltd.

### 2-2. Grouping

The mice were weighed at the introduction and classified averagely. The kinds of the experimental feeds for feeding and the number of mice per group were as follows.
Group 1: Group of ingesting low-nucleic acid feed: 8 mice
Group 2: Group of ingesting feed containing 0.6% of RNA: 8 mice
Group 3: Group of ingesting normal feed for mouse (CRF-1): 8 mice

Table 1 shows the grouping and the body weights at the introduction.

TABLE 1

| Group of Low-Nucleic | | | Group of Feed Containing | | | Group of | | |
|---|---|---|---|---|---|---|---|---|
| Acid Feed | | | 0.6% of RNA | | | Normal Feed | | |
| | Mouse | Body | | Mouse | Body | | Mouse | Body |
| Cage | ID | Weight (g) | Cage | ID | Weight (g) | Cage | ID | Weight (g) |
| 1 | #1 | 18.9 | 3 | #9 | 20.0 | 5 | #17 | 16.3 |
| | #2 | 16.6 | | #10 | 18.0 | | #18 | 19.2 |
| | #3 | 18.1 | | #11 | 16.7 | | #19 | 19.8 |
| | #4 | 18.0 | | #12 | 18.0 | | #20 | 20.0 |
| 2 | #5 | 19.4 | 4 | #13 | 19.6 | 6 | #21 | 18.8 |
| | #6 | 19.4 | | #14 | 19.6 | | #22 | 20.2 |
| | #7 | 18.3 | | #15 | 18.6 | | #23 | 17.7 |
| | #8 | 20.4 | | #16 | 18.0 | | #24 | 18.3 |

| | | | | | |
|---|---|---|---|---|---|
| Average | 18.6 | Average | 18.6 | Average | 18.8 |

### 2-3. Feed

The low-nucleic acid feed and the feed containing 0.6% of RNA were produced by CLEA Japan, Inc. The unopened feed was stored at 4° C., and the opened feed was stored at normal temperature in the breeding room. The following Table 2 shows the compositions of the low-nucleic acid feed. The feed containing 0.6% of RNA was prepared by subtracting mass % of the added yeast extract from mass % of cornstarch in the compositions shown in Table 2. The yeast extract used for the feed containing 0.6% of RNA was derived from torula yeast and was supplied by FORDAYS Co., Ltd. The yeast extract containing 70% of RNA was used for the feed containing 0.6% of RNA.

The commercially available normal for mouse CRF-1 was purchased from Oriental Yeast Co., Ltd. and used after autoclaved sterilization. Table 3 shows the data obtained from the website of Oriental Yeast Co., Ltd. in October 2020. The CRF-1 is made from brewer's yeast, corn, wheat (bran), defatted soybeans, soybeans, oil defatted rice bran, alfalfa, fish meal, and skim milk powder. Although the blending percentages of these raw materials and the RNA are not disclosed, as shown in Table 4, the CRF-1 contains RNA. This RNA is considered to be derived from each raw material.

All groups were fed with the normal feed CRF-1 during habituation.

TABLE 2

| Composition | % |
|---|---|
| Cornstarch | 45.5 |
| Vitamin-Free Casein | 24.5 |
| Granulated Sugar | 10.0 |
| Mineral Mixture (Basic Purification) | 7.0 |
| Corn Oil | 6.0 |
| Crystalline Cellulose | 3.0 |
| Cellulose Powder | 2.0 |
| Pregelatinized Starch | 1.0 |
| Vitamin Mixture (Basic Purification) | 1.0 |
| RNA | 0 |
| Total | 100.0 |

TABLE 3

| Composition | % |
|---|---|
| Water Content | 6.6 |
| Crude Protein | 17.6 |
| Crude Fat | 4.3 |
| Crude Ash | 5.1 |
| Crude Fiber | 2.3 |
| Soluble Nitrogen-Free Substance | 44.5 |
| Vitamins | 0.6 |
| Minerals | 2.7 |
| Amino Acid | 16.2 |
| Total | 100 |

### 2-4. Measurement of Amounts of Nucleic Acids in Feeds

The contents of the nucleic acids (DNA, RNA) in the low-nucleic acid feed, the feed containing 0.6% of RNA, and the normal feed were measured by the method described in Japanese Patent No. 6660994 (Japanese Patent Application No. 2018-219366, Japanese Unexamined Patent Application Publication No. 2020-085623). In detail, after that a solid feed was powdered by a crusher, a mortar, and a pestle, the feed was dissolved in a phosphate buffer solution of pH=6.0, protease treatment, protease deactivation treatment, and nuclease treatment were performed to produce a sample solution, and analysis by HPLC was performed according to the method described in Japanese Patent No. 6660994 (Japanese Patent Application No. 2018-219366, Japanese Unexamined Patent Application Publication No. 2020-085623).

Table 4 shows the total equivalent of the deoxyribonucleotides and the total equivalent of the ribonucleotides in the low-nucleic acid feed, the feed containing 0.6% of RNA, and the normal feed.

TABLE 4

| | Total Equivalent of | Total Equivalent of |
|---|---|---|
| | Deoxyribonucleotide | Ribonucleotide |
| Feed for Mouse | (g/100 g) | (g/100 g) |
| Low-Nucleic Acid Free | 0.000 | 0.019 |
| Feed Containing 0.6% of RNA | 0.000 | 0.652 |
| Normal Feed | 0.041 | 0.240 |
| (CRF-1) | | |

As the result of measurement of the amount of nucleic acid contained in the low-nucleic acid feed, the deoxyribonucleotide (that is, DNA) was equal to or less than the detection limit and the ribonucleotide (that is, RNA) was contained by the small amount, 0.019 g/100 g. On the other hand, the deoxyribonucleotide contained in the “feed containing 0.6% of RNA” produced by adding the RNA to the low-nucleic acid feed such that the amount of RNA became 0.6% was equal to or less than the detection limit and the ribonucleotide was contained by 0.652 g/100 g. The ribonucleotide in the normal feed CRF-1 was 0.240 g/100 g.

### 2-5. Preparation and Administration of ENU

An ethyl nitrosourea (ENU) test reagent from SIGMA (N8509-5G, N-Nitroso-N-ethylurea Bulk package, degree of purity of 56%) was used. The ENU was dissolved in phosphate-buffered saline (PBS), and 67.2 mg/kg of the body weight was administered intraperitoneally

### 2-6. Measurement of Food Intake

The bedding was changed on the previous day of the measurement of food intake, and weights of feeders containing the experimental feeds were weighed. The weights of the feeders containing the experimental feeds and the weights of the experimental feeds that had dropped on the beddings were measured after about 24 hours. The food intake per cage was calculated from the difference between the sum of both weights and the weight of the feeder on the previous day. The food intake per mouse was calculated by dividing the food intake per cage by the number of mice housed. The similar work was performed for 3 consecutive days.

Table 5 shows the results of the food intake measurement for three days from day 7 of feeding the experimental feeds.

TABLE 5
Measurement of Food Intake (1)

| | | Number | Food Intake (g/Mouse) | | | |
|---|---|---|---|---|---|---|
| | Cage | of Mice | Day | Day | Day | Average |
| Group | No. | Housed | 1 | 2 | 3 | in Group |
| Low-Nucleic | 1 | 4 | 2.23 | 2.43 | 2.85 | 2.23 |
| Acid Feed | 2 | 4 | 1.40 | 2.05 | 2.45 | |
| Feed Containing | 3 | 4 | 2.35 | 2.38 | 2.95 | 2.61 |
| 0.6% of RNA | 4 | 4 | 2.48 | 2.53 | 2.98 | |
| Normal Feed | 5 | 4 | 3.15 | 3.15 | 3.75 | 3.29 |
| | 6 | 4 | 3.15 | 3.10 | 3.45 | |

Table 6 shows the results of the food intake measurement for three days from day 20 of ENU administration.

TABLE 6
Measurement of Food Intake (2)

| | | Number | Food Intake (g/Mouse) | | | |
|---|---|---|---|---|---|---|
| | Cage | of Mice | Day | Day | Day | Average |
| Group | No. | Housed | 1 | 2 | 3 | in Group |
| Low-Nucleic | 1 | 4 | 1.96 | 2.71 | 1.51 | 2.24 |
| Acid Feed | 2 | 4 | 2.52 | 2.48 | 2.27 | |
| Feed Containing | 3 | 4 | 3.02 | 2.57 | 2.36 | 2.57 |
| 0.6% of RNA | 4 | 4 | 2.62 | 2.14 | 2.72 | |
| Normal Feed | 5 | 4 | 3.39 | 3.31 | 3.28 | 3.25 |
| | 6 | 4 | 3.16 | 3.24 | 3.13 | |

Although the food intakes of the experimental feeds were measured before and after the ENU administrations, there were no differences in food intake between the group of low-nucleic acid feed and the group of feed containing 0.6% of RNA. In the normal feed, the food intake was larger than those of the group of low-nucleic acid feed and the group of feed containing 0.6% of RNA. It is presumed that this occurred due to the difference in the hardness and the basic composition of the feeds.

### 2-7. Measurement of Body Weight

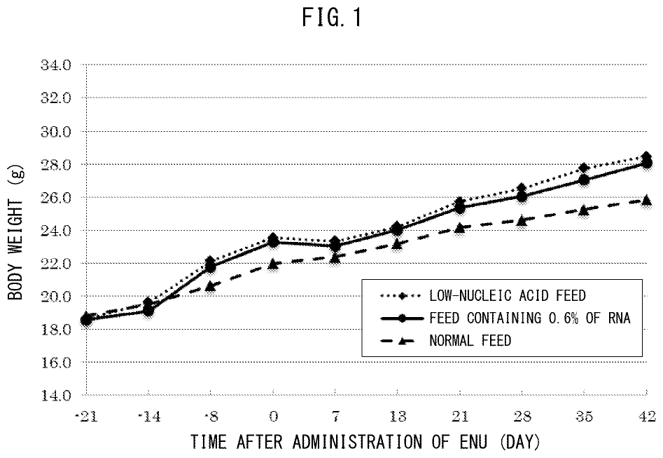

FIG. 1 illustrates the results of measurement of the body weights weekly between 1:00 p.m. to 2:00 p.m. after the start of feeding the experimental feeds at the time of introduction of the mice. There was no difference between the group of low-nucleic acid feed and the group of feed containing 0.6% of RNA. The group of normal feed resulted in the body weight lower than the others. It is presumed that this occurred due to the difference in the basic composition of the experimental feeds.

### 3. Pig-a Assay Test Method

### 3-1 Principle

The Pig-a assay is a method that allows analyzing gene mutations using a small amount of peripheral blood and a flow cytometer. When a mutation occurs in a Phosphatidylinositol glycan anchor biosynthesis, class A (Pig-a) gene, the cells lose GPI anchor. The GPI anchor serves as tethering various proteins to surfaces of cell membranes. Originally, CD24, which is GPI anchor binding protein, or the like is presented on a surface of a cell membrane of a red blood cell. However, CD24 protein is not presented on a red blood cell where Pig-a gene mutation occurs. Using the property, the red blood cells are stained with a fluorescence-labeled antibody with respect to CD24 protein, and the number of red blood cells where the CD24 protein is presented on the surfaces of the cell membranes and the number of red blood cells where the CD24 protein is not presented are measured by the flow cytometer, thus allowing measuring the frequency of mutations of the Pig-a gene.

### 3-2. Blood Collection

The mouse was placed in a fixator and the tail was disinfected with 70% alcohol. A 23G ingestion needle was inserted into the tail vein to bleed, 4 μL of the blood was immediately collected with a pipette and was mixed with 1 μL of EDTA. The blood can also be collected by incising a portion of the tail vein with a blade of a razor for bleeding.

### 3-3 Adjustment and Staining of Blood Samples

Prior to staining, 100 μL of saline with 1% of fetal bovine serum, 3 μL of PE/Cy7 anti-mouse TER-119/Erythroid Cells (BioLegend), and 2 μL of FITC anti-mouse CD24 (BioLegend) are mixed and an antibody-stained solution is produced. 1 μL of the collected blood sample was added to 150 μL of saline with serum, the product was mixed well and then centrifuged at 1000×g at 4° C. for 5 minutes. After removal of the supernatant, the product was suspended in 100 μL of an antibody staining solution and stained by leaving it for 40 minutes in a dark place at 4° C. After the stained blood samples were stirred, it was centrifuged at 1000×g at 4° C. for 5 minutes. The supernatant was removed and suspended in 500 μL of saline with serum. Note that the samples can be used for measurement as long as they were stained within 24 hours, and are stored in a cool and dark place until immediately before measurement.

### 3-4 Measurement with Flow Cytometer

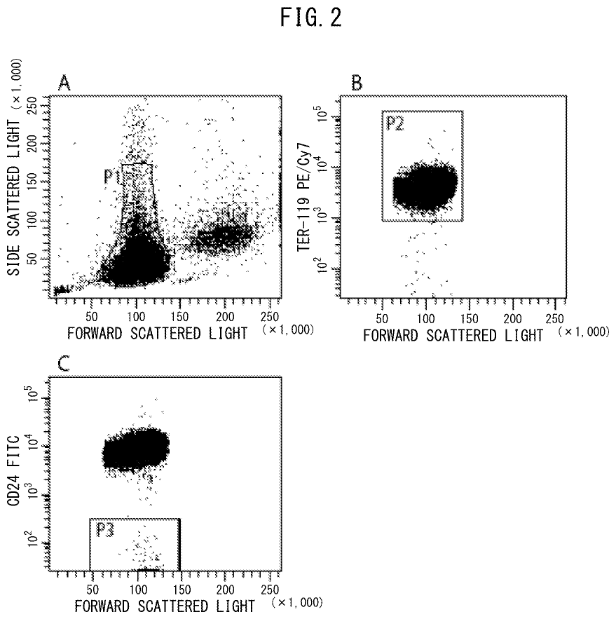

The stained blood samples were measured with the flow cytometer Gallios (Beckman Coulter) or FACS CantoII (BD FACS). FIG. 2 shows examples of the results of Pig-a assay in which the flow cytometer measured the blood after 43 days of the ENU administration. First, a gate P1 was set to a single cell population on a plot of forward scattered light and side scattered light (an operation of selecting a specific cell population in the sample as an analysis target on the data) (FIG. 2-A). Further, a gate P2 was set to a red blood cell marker TER-119 positive cell population in the single cell population (gate P1) using a fluorescence of PE/Cy7 labeled with an anti-TER-119 antibody as an index (FIG. 2-B). Next, a gate P3 was set to the part of CD24 negative in the red blood cell population (gate P2) using a fluorescence of FITC labeled with anti-CD24 antibody as an index (FIG. 2-C). The number of cells that were CD24 negative (gate P3) was counted in the red blood cell population (gate P2), and the number of CD24 negative cells (which is equal to the number of cells in the gate P3, namely, the number of Pig-a gene mutation red blood cells) was divided by the number of TER-119 positive cells (the number of cells in the gate P2, namely, the total number of measured red blood cells) to calculate the frequency of the Pig-a gene mutations. Note that the measurement was performed such that the number of red blood cells (TER-119 positive cells) was approximately 1 million in each sample.

In the Pig-a assay using the blood of the mouse, as shown in of FIG. 2-C, the many red blood cells that were CD24 negative (gate P3) were observed in the blood of the mouse administered with mutagenic ENU compared with blood of the mouse administered with PBS as the negative contrast (the data is not shown), and induction of gene mutations can be detected.

### 4. Results of Pig-a Assay

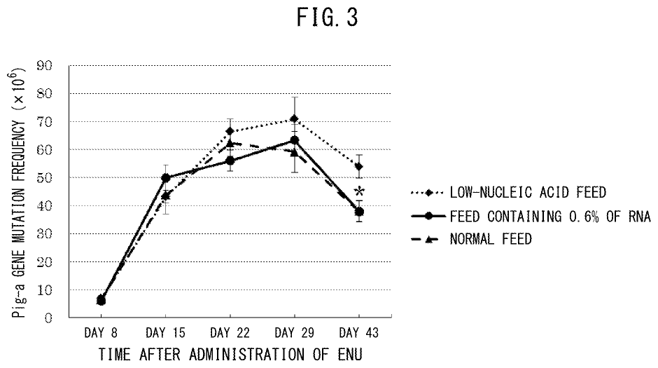

FIG. 3 shows a graph of measuring the frequencies of gene mutations over time after the ENU was administered with the mouse that had ingested the low-nucleic acid feed, the mouse that had ingested the feed containing 0.6% of RNA, and the mouse that had ingested the normal feed by Pig-a assay.

The mouse that had ingested the low-nucleic acid feed, the mouse that had ingested the feed containing 0.6% of RNA, and the mouse that had ingested the normal feed had no difference in the frequency of Pig-a gene-mutations at day 8 or day 15 after the ENU administration. After that, breeding with each of the experimental feeds was continued ongoingly. The mouse that had ingested the feed containing 0.6% of RNA and the mouse that had ingested the normal feed tended to decrease in the frequency of gene mutations compared with the mouse that had ingested the low-nucleic acid feed at day 22 and day 29 after the ENU administration. At day 43 after the ENU administration, the mouse that had ingested the feed containing 0.6% of RNA and the mouse that had ingested the normal feed significantly decreased in the frequency of gene mutations compared with the mouse that had ingested the low-nucleic acid feed.

Regarding the results of this test, given that ingestion of the yeast extract or the like brings a defense action against DNA damage itself, the difference between the ingestion group and the non-ingestion group of the yeast extract or the like occurs at the early stage after the ENU administration. However, the gene mutations occurred in both groups to the same extent from day 8 to day 15 after the ENU administration, and therefore it is determined that the active site of the yeast extract or the like is not the defense action against DNA damage. Therefore, intake of the yeast extract or the like (particularly RNA) is considered to promote the repair of DNA to suppress gene mutations, not the defense action against the DNA damage itself. On the other hand, when significant DNA damage occurs, the cells are eliminated by cell death and the like to reduce the accumulation of gene mutations. Even under a condition accompanied by strong DNA damage beyond the repair ability, the ingestion of the yeast extract or the like (particularly RNA) is considered to eventually reduce the accumulation of gene mutations.

1. A drug for suppressing a gene mutation comprising:
a yeast extract, wherein
RNA included in the yeast extract is contained as an active ingredient.

2. The drug for suppressing the gene mutation according to claim 1, further comprising:
one or more of components derived from a plant, fish, and a dairy product in addition to the yeast extract, wherein
RNA is contained as an active ingredient.

3. The drug for suppressing the gene mutation according to claim 1, wherein
the RNA promotes an action of repairing damaged DNA or promotes an action of killing cells containing the damaged DNA.

4. The drug for suppressing the gene mutation according to claim 1, wherein
the RNA is contained by 0.08 to 20 mass %.

5. The drug for suppressing the gene mutation according to claim 1, wherein
the RNA contained in the yeast extract is RNA extracted from a torula yeast.

6. A health food for suppressing a gene mutation, comprising:
a yeast extract, wherein
RNA included in the yeast extract is contained as an active ingredient.

7. The health food for suppressing the gene mutation according to claim 6, further comprising
one or more of components derived from a plant, fish, and a dairy product in addition to the yeast extract, wherein
RNA is contained as an active ingredient.

8. The health food for suppressing the gene mutation according to claim 6, wherein
the RNA promotes an action of repairing damaged DNA or promotes an action of killing cells containing the damaged DNA.

9. The health food for suppressing the gene mutation according to claim 6, wherein
the RNA is contained by 0.08 to 20 mass %.

10. The health food for suppressing the gene mutation according to claim 6, wherein
the RNA contained in the yeast extract is RNA extracted from a torula yeast.
